(19) United States
(12) Patent Application Publication
(10) Pub. No.: US 2016/0332932 A1
(43) Pub. Date: Nov. 17, 2016

(54) PROCESSES FOR REDUCING DIENES IN A HIGH PURITY POLYENE STREAM

(71) Applicant: UOP LLC, Des Plaines, IL (US)

(72) Inventors: Joseph A. Montalbano, Elmhurst, IL (US); Richard S. Kempf, Deerfield, IL (US)

(21) Appl. No.: 14/711,242

(22) Filed: May 13, 2015

(51) Int. Cl. C07C 7/163; C07C 7/167

(52) U.S. Cl. CPC C07C 7/163; C07C 7/167

(57) ABSTRACT

Processes for producing a propylene stream having at least 95% propylene and a reduced level of dienes. A selective hydrogenation zone converts dienes into olefins in a stream having at least C3 and C4 olefins from a feed stream including butene and C4 dienes. The selective hydrogenation zone may be disposed between a demethanizer and a deethanzier or it may be disposed downstream of both the demethanizer and the deethanizer. A C3 splitter and an acetylene conversion zone may be utilized.

### FIELD OF THE INVENTION

This invention relates generally to processes which provide a high purity propylene product, and more particularly to such processes which include a selective hydrogenation to reduce the amount of dienes in the high purity propylene product.

### BACKGROUND OF THE INVENTION

Propylene demand in the petrochemical industry has grown substantially, largely due to its use as a precursor in the production of polypropylene for packaging materials and other commercial products. Other downstream uses of propylene include the manufacture of acrylonitrile, acrylic acid, acrolein, propylene oxide and glycols, plasticizer oxo alcohols, cumene, isopropyl alcohol, and acetone, to name a few.

Propylene has typically been produced during the steam cracking or pyrolysis of hydrocarbon feedstocks such as natural gas, petroleum liquids, and carbonaceous materials (e.g., coal, recycled plastics, and organic materials), to produce ethylene. Additional significant sources of propylene are byproducts of fluid catalytic cracking (FCC) and residue fluid catalytic cracking (RFCC), normally targeting gasoline production. FCC is described, for example, in U.S. Pat. No. 4,288,688 and elsewhere. A mixed, olefinic C3/C4 byproduct stream of FCC may be purified in propylene to polymer grade specifications by the separation of C4 hydrocarbons, propane, ethane, and other compounds.

More recently, the desire for propylene and other light olefins from alternative, non-petroleum based feeds has led to the use of oxygenates such as alcohols and, more particularly, methanol, ethanol, and higher alcohols or their derivatives. Methanol, in particular, is useful in a methanol-to-olefin (MTO) conversion process described, for example, in U.S. Pat. No. 5,914,433. The yield of light olefins from such a process may be improved using olefin cracking to convert some or all of the C4+ product of MTO in an olefin cracking reactor, as described in U.S. Pat. No. 7,268,265. Other processes for the targeted production of light olefins involve high severity catalytic cracking of naphtha and other hydrocarbon fractions. A catalytic naphtha cracking process of commercial importance is described in U.S. Pat. No. 6,867,341.

Paraffin dehydrogenation represents yet another dedicated route to light olefins and is described in U.S. Pat. No. 3,978,150 and elsewhere. However, the capital cost associated with a propane dehydrogenation plant is normally justified only in cases of large-scale propylene production units (e.g., typically 250,000 metric tons per year or more). The substantial supply of propane feedstock required to main this capacity is typically available from propane-rich liquefied petroleum gas (LPG) streams from gas plant sources.

In the current designs, a propylene product, from any source, was purified and separated into a stream comprising greater than 99% propylene. The separation and purification of the propylene product from other components is typically accomplished through various fractionization columns in which the components are separated based upon different boiling points. One column that is often utilized in such a separation process is a C3 splitter column. The C3 splitter column separates the propylene product from propane, C4 dienes, such as methyl acetylene and propadiene, and C4+ hydrocarbons. The C3 splitter column is a large column that requires a large energy input to operate and separate the various components.

While these processes are effective at providing a very high level of purity for a propylene stream, in some instances a producer may require only greater than 95% propylene, so long as dienes, such as methyl acetylene and propadiene are below a certain level. Indeed, some processes may produce a stream comprising approximately 95%, however, the amount of the dienes in the stream may be approximately 250 ppm. If the amount of dienes may be lowered, it is believed that a propylene stream having a sufficient level of purity, but below the 99+%, can be utilized by some processors. Lowering the amount of dienes in the stream via the C3 splitter column would require a significant amount of energy input, at considerable expense to the processor.

Therefore, it would be desirable to have one or more processes for producing a propylene product stream with a lower amount of dienes which did not require a C3 splitter column to lower the diene level.

### SUMMARY OF THE INVENTION

One or more processes have been invented for providing a high purity propylene stream in which a selective hydrogenation zone is used to convert dienes to olefins. As a result, a high purity propylene stream (meaning greater than or equal to 95% propylene) can be produced without necessarily including a C3 splitter column.

Therefore, in a first aspect of the present invention, the present invention may be characterized broadly as providing a process for producing a purified propylene stream by: separating a propylene rich stream comprising at least C3 and C4 olefins from at least a portion of a C4− olefins stream, the C4− olefins stream including dienes and acetylenes; selectively hydrogenating the dienes in the propylene rich stream to provide a partially hydrogenated effluent stream; separating a high purity propylene stream (at least 95%) from at least a portion of the partially hydrogenated effluent stream.

In at least one embodiment of the present invention, the high purity propylene stream comprises less than or equal to 10 ppm of methyl acetylene plus propadiene.

In some embodiments of the present invention, the process further includes separating the partially hydrogenated effluent stream into a C2− stream and a bottoms stream. The high purity propylene stream may be separated from the bottoms stream. It is contemplated that the process includes hydrogenating acetylene in the C2− stream to provide an acetylene lean stream. It is further contemplated that the process includes separating the acetylene lean stream into at least an ethylene stream and an ethane stream. It is even further contemplated that the ethylene stream comprises less than or equal to 1 ppm of acetylene.

In various embodiments of the present invention, the process further includes compressing a feed stream to provide a compressed feed stream, wherein the C4− olefins stream comprises a portion of the compressed feed stream; and, separating a light ends stream from the compressed feed stream to provide the C4− olefins stream.

In one or more embodiments of the present invention, the propylene rich stream is separated from the at least a portion of the C4− olefins stream in a separation zone configured to provide a C2− stream and a C3+ stream, the C3+ stream comprising the propylene rich stream.

In one or more embodiments of the present invention, the propylene rich stream is separated from the at least a portion of the C4− olefins stream in a separation zone configured to provide a lights ends stream and a C2+ stream, the C2+ stream comprising the propylene rich stream. It is contemplated that the process also includes hydrogenating acetylene in the C2+ stream in a hydrogenation zone and selectively hydrogenating the dienes in the propylene rich stream occurs in the same hydrogenation zone.

In a second aspect of the present invention, the present invention may be generally characterized as providing a process for producing a purified propylene stream by: separating a C2-4 olefins stream from a feed stream comprising butene and C4 dienes in a first separation zone; passing the C2-4 olefins stream to a selective hydrogenation zone having a reactor and being operated to convert dienes into olefins and provide a partially hydrogenated effluent stream; and, passing at least a portion of the partially hydrogenated effluent stream to a second separation zone having a fractionation column configured to provide a high purity propylene stream and a C4+ stream.

In one or more embodiments of the present invention, the process includes passing the feed stream to a compression zone configured to provide a compressed feed stream, and, passing the compressed feed stream to the first separation zone configured to separate a light ends stream from the compressed feed stream and provide the C2-4 olefins stream.

In at least one embodiment of the present invention, the process includes heating the C2-4 olefins stream with the partially hydrogenated effluent stream.

In various embodiments of the present invention, the process further includes passing the partially hydrogenated effluent stream to a third separation zone having a fractionation column being configured to provide a C2− stream and a bottoms stream, and, passing the bottoms stream from the third separation zone to the second separation zone. It is contemplated that the process also includes passing the C2− stream to an acetylene conversion zone configured to convert acetylene in the C2− stream and provide an acetylene lean stream. It is further contemplated that the process also includes removing oxygenates from the acetylene lean stream to provide a purified stream. It is also contemplated that the process also includes passing the purified stream to a fourth separation zone configured to provide at least an ethylene stream and an ethane stream. It is even further contemplated that the ethylene stream comprises less than or equal to 1 ppm of acetylene. It is also further contemplated that the feed stream comprises an effluent from a reaction zone. It is further contemplated that the fourth separation zone also provides a recycle gas stream comprising hydrogen and ethylene, and the process further includes recycling the recycle gas stream to the reaction zone.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

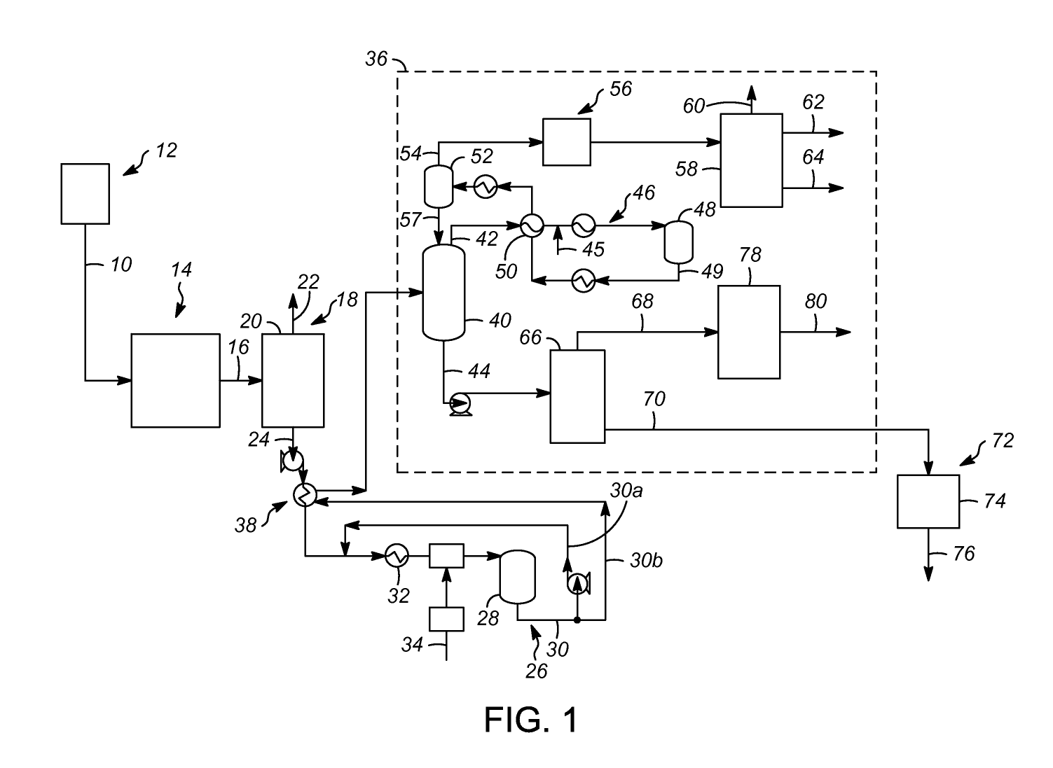

FIG. 1 shows a process flow diagram according to one or more embodiments of the present invention; and,

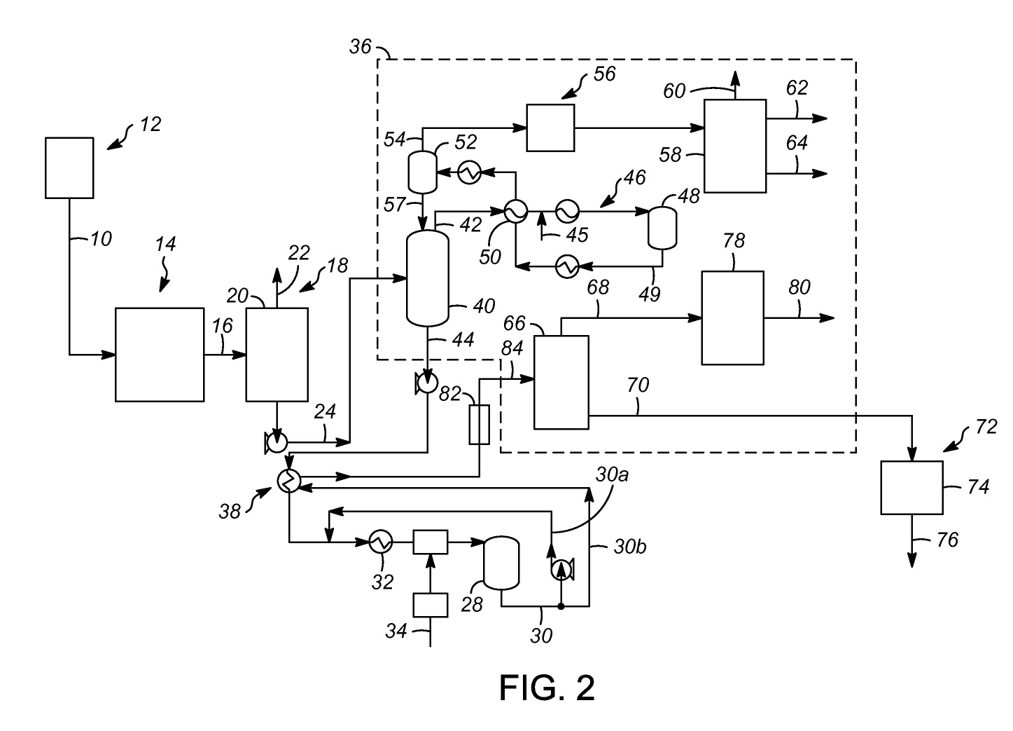

FIG. 2 shows another process flow diagram according to one or more embodiments of the present invention.

### DETAILED DESCRIPTION OF THE INVENTION

As mentioned above, one or more processes have been invented for providing a high purity propylene stream in which a selective hydrogenation zone is used to convert dienes to olefins. In some instances, the a propylene stream having greater than or equal to 95% propylene can be separated from a feed without the need for a C3 splitter column. Such streams however, may comprise approximately 250 ppm of methyl acetylene and propadiene. Rather than separate the propylene and these dienes via a fractionation in a C3 splitter column, the various embodiments of the present invention propose to selectively hydrogenate these dienes in order to lower the concentration within the propylene stream. A high purity propylene stream can be produced, and if desired, passed to a C3 splitter column, although in some applications doing so may not be necessary. The selective hydrogenation, in some instances, can also replace an acetylene conversion zone. In at least one embodiment, the selective hydrogenation is done downstream of a deethanizer. In other embodiments, the selective hydrogenation is done upstream of a deethanizer and downstream of a demethnaizer. In any embodiment, the various processes will provide a high purity propylene stream with a lower amount of dienes. The use of the selective hydrogenation is less costly than the C3 splitter, allowing a refiner to efficiently and economically produce a useable high purity propylene stream.

With these general principles in mind, one or more embodiments of the present invention will be described with the understanding that the following description is not intended to be limiting.

As shown in FIGS. 1 and 2, a feed stream 10 comprises a portion of an effluent from a reaction zone 12. The feed stream 10 comprises C1-C4 paraffins, C2-C4 dienes, hydrogen, nitrogen, carbon oxides, and other components and is predominately (i.e., more than 50%) propylene. The reaction zone 12 may comprise any suitable reaction zone 12 for provide the feed stream 10 for example, an oxygenate conversion zone.

As is known, in an oxygenate conversion zone, an oxygenate feed, e.g., methanol, is contacted with a molecular sieve catalyst, usually a silicoaluminophosphate (SAPO) molecular sieve catalyst, under conditions designed to convert the oxygenate feed into predominately light olefins. As used herein, references to “light olefins” are to be understood to generally refer to C2 and C3 olefins, i.e., ethylene and propylene, alone or in combination. In particular, the oxygenate conversion reactor section produces or results in formation of an oxygenate conversion reactor effluent stream which generally comprises fuel gas hydrocarbons such as methane, ethane and propane, light olefins, and C4+ hydrocarbons. A non-limiting list of suitable SAPO molecular sieve catalysts includes SAPO-17, SAPO-18, SAPO-34, SAPO-35, SAPO-44, and mixtures thereof. The equipment and conditions with which this conversion reaction is conducted are well known to those skilled in the art and do not need to be detailed here. Numerous patents describe this process for various types of these catalysts including U.S. Pat. No. 3,928,483; U.S. Pat. No. 4,025,575; U.S. Pat. No. 4,252,479; U.S. Pat. No. 4,496,786; U.S. Pat. No. 4,547,616; U.S. Pat. No. 4,677,242; U.S. Pat. No. 4,843,183; U.S. Pat. No. 4,499,314; U.S. Pat. No. 4,447,669; U.S. Pat. No. 5,095,163; U.S. Pat. No. 5,191,141; U.S. Pat. No. 5,126,308; U.S. Pat. No. 4,973,792; and U.S. Pat. No. 4,861,938, the disclosures of which are incorporated herein by reference. In general, the process for converting an oxygenate feedstock in the presence of a molecular sieve catalyst can be carried out in a variety of reactors, including as representative examples a fixed bed process, a fluidized bed process (includes a turbulent bed process), a continuous fluidized bed process, and a continuous high velocity fluidized bed process. In addition to light olefins, an effluent stream from the oxygenate conversion zone also typically includes methane, ethane, propane, DME, C4 olefins and saturates, C5+ hydrocarbons, water and other hydrocarbon components in minor amount.

Returning to FIGS. 1 and 2, in various embodiments, the feed stream 10 may be passed to a compression zone 14 in which the feed stream 10 is compressed in one or more stages (e.g., in one or more compressors) to form a compressed feed stream 16. Typically, after each stage of compression, the compressed stream is cooled causing the condensation of heavier components which can be collected in one or more knock out drums between compression stages (not shown).

From the compression zone 14, the compressed feed stream 16 is passed to a separation zone 18 comprising a demethanizer column 20. In the demethanizer column 20, the compressed feed stream 16 is fractionated, such as by conventional distillation, to provide a demethanizer overhead stream 22 predominantly comprising a light ends stream or C1-hydrocarbons including methane, and also comprising hydrogen, carbon oxides, and nitrogen and a demethanized C2+ bottoms stream 24 comprising predominately propylene, and also comprising ethylene, ethane, C4− dienes and acetylene. In one or more embodiments of the present invention, the demethanized C2+ bottoms stream 24 comprises a propylene rich steam as the concentration of propylene in the demethanized C2+ bottoms stream 24 is higher compared to the concentration in the compressed feed stream 16.

With reference to FIG. 1, in order to reduce the dienes in the demethanized C2+ bottoms stream 24, the demethanized C2+ bottoms stream 24 is subjected to a selective hydrogenation in a selective hydrogenation zone 26 having a selective hydrogenation reactor 28 configured and operated to catalytically convert methyl acetylene and butadiene to butenes. A stream containing C4 hydrocarbons, in the depicted embodiments, the demethanized C2+ bottoms stream 24, is passed to the selective hydrogenation reactor 28 to convert diolefins, and especially butadienes, to olefins, and to provide a partially hydrogenated effluent stream 30.

As shown in FIG. 1, the demethanized C2+ bottoms stream 24 is first heated in at least one heat exchanger 32 (discussed below), then mixed with a hydrogen containing gas 34, and then the demethanized C2+ bottoms stream 24, along with the hydrogen, is passed into the selective hydrogenation reactor 28. Other processes may be employed.

Conditions and catalysts to employ in the selective hydrogenation reactor 28 will be recognized by those skilled in the art. A representative selective hydrogenation process for converting diolefins to monoolefins is described, for example, in U.S. Pat. No. 4,695,560, with respect to a selective hydrogenation catalyst comprising nickel and sulfur dispersed on an alumina support material having a high surface area. Selective hydrogenation is normally performed with a selective hydrogenation zone 26 being maintained under relatively mild hydrogenation conditions, including an absolute pressure from about 280 kPa (40 psia) to about 5500 kPa (800 psia), with a range from about 350 kPa (50 psia) to about 2100 kPa (300 psia) being preferred. Relatively moderate selective hydrogenation zone temperatures, for example, from about 25° C. (77° F.) to about 350° C. (662° F.), preferably from about 50° C. (122° F.) to about 200° C. (392° F.), are representative. The liquid hourly space velocity (LHSV) is typically greater than about 1 hr⁻¹, and preferably greater than about 5 hr⁻¹ (e.g., between about 5 and about 35 hr⁻¹). The LHSV, closely related to the inverse of the reactor residence time, is the volumetric liquid flow rate over the catalyst bed divided by the bed volume and represents the equivalent number of catalyst bed volumes of liquid processed per hour. An important variable in selective hydrogenation is the ratio of makeup hydrogen to diolefins in the hydrocarbon feed to the selective hydrogenation process. To avoid the undesired saturation of a significant proportion of the monoolefins, generally less than about 2 times the stoichiometric hydrogen requirement for diolefin saturation is used. Selective hydrogenation therefore requires the addition of makeup hydrogen that can have varying levels of purity, depending on the source.

In addition to converting the dienes to monoolefins or paraffins, in the selective hydrogenation zone 26, acetylene may be converted into ethylene or ethane. However, this is merely one embodiment, and other embodiments may still include an acetylene conversion zone.

With a lower concentration of dienes, a high purity propylene stream may be separated from at least a portion of the partially hydrogenated effluent stream 30 in a separation zone 36 having one or more columns and/or vessels. A preferred separation zone is shown in FIG. 1.

More particularly, a portion 30a of the partially hydrogenated effluent stream 30 may be used as a diluent recycle to the selective hydrogenation zone 26 to ensure that the stoichiometric hydrogen requirement within the selective hydrogenation reactor 28 is in the desired range. The remaining portion 30b of the partially hydrogenated effluent stream 30 may pass through a heat exchanger 38 to provide heat to the demethanized C2+ bottoms stream 24 which is entering the selective hydrogenation zone 26. From the heat exchanger 38, the partially hydrogenated effluent stream 30 may be passed to a fractionation column, such as a deethanizer column 40.

In the deethanizer column 40, the partially hydrogenated effluent stream 30 is fractionated, such as by conventional distillation, to provide a deethanizer overhead stream 42 comprising C2 and lighter hydrocarbons (i.e., C2− hydrocarbons, including hydrogen, methane, acetylene, ethane, ethylene) and a deethanized C3+ bottoms stream 44 comprising predominately compounds heavier than ethane, such as propylene, propane, mixed butenes and/or butane. The deethanizer overhead stream 42 comprising C2 and lighter hydrocarbons may be refined to recover one or more product streams, such as an ethylene stream.

If the selective hydrogenation zone 26 was not operated to convert acetylene to ethylene or ethane, the deethanizer overhead stream 42 may be combined with a hydrogen containing gas 45 and then passed to an acetylene conversion zone 46 having an acetylene conversion reactor 48. In the acetylene conversion reactor 48, acetylene is selectively converted into ethylene or ethane. The conditions of such an acetylene conversion zone 46 are known to those of ordinary skill in the art.

An effluent stream 49 from the acetylene conversion zone 46 may be heated in a reboiler 50 and passed to a receiver 52 which will separate the effluent stream 49 into a vapor stream 54 and a liquid reflux stream 57 which is passed back to the deethanizer column 40. The vapor stream 54 from the receiver 52 may be passed to a guard bed zone 56 to remove any dimethyl ether (DME) and other trace oxygenates before being passed to a C2 splitter column 58.

In the C2 splitter column 58, the vapor stream 54 from the receiver 52 is treated, e.g., is fractionated, such as by conventional distillation, to provide an overhead recycle stream 60 comprising hydrogen and some trace amounts of ethylene, a sidecut stream 62 comprising an ethylene product stream and a bottoms stream 64 principally comprising ethane. With acetylene conversion, either in the selective hydrogenation zone 26 or in the acetylene conversion zone 46, the ethylene product stream 62 may comprises less than or equal to 1 ppm of acetylene. The ethane-containing bottoms stream 64, or a portion thereof can be used as fuel. The overhead stream 60 may be recycled back (not shown) to the reaction zone 12.

Returning to the deethanizer column 40, the deethanized C3+ bottoms stream 44 or at least a portion thereof, may be passed to a depropanizer column 66. In the depropanizer column 66, the deethanized C3+ bottoms stream 44 can be treated, or fractionated, such as by conventional distillation, to produce a depropanizer overhead stream 68 comprising a high purity propylene stream and a depropanized stream 70 generally comprising C4+ components.

At least a portion of the depropanized stream 70, the C4+ stream, can be processed in an olefin cracking zone 72 in order to increase the production of light olefins, particularly propylene. The olefin cracking zone 72 comprises an olefin cracking reactor (OCR) 74 provides a way for increasing the overall yield of light olefin from an oxygenate feed and thus is particularly desirable when the reaction zone 12 comprises an MTO reaction zone.

The design and conditions of operation of the olefin cracking reactor 74, including the selection of a suitable catalyst, are well understood by those skilled in the art. U.S. Pat. No. 6,646,176, the description of which is incorporated herein by reference, exemplifies suitable catalysts and operating conditions. Other catalysts and operating parameters will be recognized by those skilled in the art and the present invention is not limited to any particular method. Generally, the olefin cracking reactor 74 converts larger olefins, including C4 olefins and larger hydrocarbons, including higher olefins and paraffins, to light olefins, primarily propylene. The production of light olefins from the olefin cracking reactor does not consume ethylene. An effluent stream 76 from the olefin cracking reactor can be recycled back to allow for recovery of the lighter olefins including propylene.

Returning to the depropanizer column 66, the high purity propylene stream 68 comprises at least 95% propylene and less than or equal to 10 ppm of methyl acetylene plus propadiene. In some embodiments, this level of purity is sufficient, and no further refinement is required. However, it is contemplated that the high purity propylene stream 68 is passed to a C3 splitter column 78 to provide a propylene stream 80 that has a purity greater than 99% propylene. However, the C3 splitter column 78 does not require as much energy input to separate the components to provide a propylene stream 80 that has a purity greater than 99%. Thus, even if the C3 splitter column 78 is utilized, the energy consumption will be lowered and may lead to utility savings.

Turning to FIG. 2, in another embodiment of the present invention, the selective hydrogenation zone is disposed downstream of the deethanizer column.

As shown in this embodiment of the present invention, the demethanized C2+ bottoms stream 24 is passed to the deethanizer column 40, which will again provide the deethanizer overhead stream 42 comprising C2 and lighter hydrocarbons (i.e., C2− hydrocarbons, including hydrogen, methane, acetylene, ethane, ethylene), and the deethanized C3+ bottoms stream 44 comprising predominately compounds heavier than ethane, such as propylene, propane, mixed butenes, butane, and dienes such as methyl acetylene and propadiene.

The processing of the deethanizer overhead stream 42 is the same in this embodiment as show in FIG. 1, and that description is incorporated herein by reference. However, since the deethanizer overhead stream 42 separated by the deethanizer column 40 not yet been subjected to selective hydrogenation, it is contemplated for this embodiment, that the deethanizer overhead stream 42, in most embodiments, will be passed to an acetylene conversion unit.

As shown in FIG. 2, the deethanized C3+ bottoms stream 44 will comprise a propylene rich stream as the deethanized C3+ bottoms stream 44 has a greater concentration compared to the demethanized C2+ bottoms stream 24. Accordingly, the demethanized C2+ bottoms stream 44 is passed to the selective hydrogenation zone 26, which may be the same in this embodiment as show in FIG. 1, and that description is incorporated herein by reference. However, since the deethanized C3+ bottoms stream 44 is lean in acetylene, it is unlikely that acetylene conversion will occur.

As with the above described embodiment, a portion 30a of the partially hydrogenated effluent stream 30 from the selective hydrogenation zone 26 may be recycled back to the selective hydrogenation zone 26 as a recycle stream, while the remainder may be used to heat the deethanized C3+ bottoms stream 44 in the heat exchanger 38.

In order to remove any trace amounts of hydrogen from the partially hydrogenated effluent stream 30, the partially hydrogenated effluent stream 30 is passed to a hydrogen separation zone 82 which separates hydrogen from the heavier olefins. Exemplary separation techniques include hydrogen strippers or PSA (Pressure Swing Adsorption) units. Such units are well known in the art. A hydrogen lean partially hydrogenated effluent stream 84 may be passed from the hydrogen separation zone 82 to the depropanizer column 66. In the depropanizer column 66, the hydrogen lean partially hydrogenated effluent stream 84 will be separated and processed in the same manner as described above with respect to FIG. 1.

In either embodiment, by selectively lowering an amount of dienes in a propylene rich stream by selective hydrogenation, a propylene stream comprising greater than 95% propylene and less than 10 ppm of methyl acetylene plus propadiene may be efficiently recovered. In cases where the product can have such a level of purity, the recovery may be more economical for a refiner, leading to a cost savings.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing a purified propylene stream, the process comprising:
separating a propylene rich stream comprising at least C3 and C4 olefins from at least a portion of a C4− olefins stream, the C4− olefins stream including dienes and acetylenes;
selectively hydrogenating the dienes in the propylene rich stream to provide a partially hydrogenated effluent stream;
separating a high purity propylene stream from at least a portion of the partially hydrogenated effluent stream.

2. The process of claim 1 wherein the high purity propylene stream comprises less than or equal to 10 ppm of methyl acetylene plus propadiene.

3. The process of claim 1 further comprising:
separating the partially hydrogenated effluent stream into a C2− stream and a bottoms stream, wherein the high purity propylene stream is separated from the bottoms stream.

4. The process of claim 3 further comprising:
hydrogenating acetylene in the C2− stream to provide an acetylene lean stream.

5. The process of claim 4 further comprising:
separating the acetylene lean stream into at least an ethylene stream and an ethane stream.

6. The process of claim 5 wherein the ethylene stream comprises less than or equal to 1 ppm of acetylene.

7. The process of claim 1, further comprising:
compressing a feed stream to provide a compressed feed stream, wherein the C4− olefins stream comprises a portion of the compressed feed stream; and,
separating a light ends stream from the compressed feed stream to provide the C4− olefins stream.

8. The process of claim 1 wherein the propylene rich stream is separated from the at least a portion of the C4− olefins stream in a separation zone configured to provide a C2− stream and a C3+ stream, the C3+ stream comprising the propylene rich stream.

9. The process of claim 1 wherein the propylene rich stream is separated from the at least a portion of the C4− olefins stream in a separation zone configured to provide a lights ends stream and a C2+ stream, the C2+ stream comprising the propylene rich stream.

10. The process of claim 9 further comprising:
hydrogenating acetylene in the C2+ stream in a hydrogenation zone, wherein selectively hydrogenating the dienes in the propylene rich stream occurs in the same hydrogenation zone.

11. A process for producing a purified propylene stream, the process comprising:
separating a C2-4 olefins stream from a feed stream comprising butene and C4 dienes in a first separation zone;
passing the C2-4 olefins stream to a selective hydrogenation zone having a reactor and being operated to convert dienes into olefins and provide a partially hydrogenated effluent stream; and,
passing at least a portion of the partially hydrogenated effluent stream to a second separation zone having a fractionation column configured to provide a high purity propylene stream and a C4+ stream.

12. The process of claim 11 further comprising:
passing the feed stream to a compression zone configured to provide a compressed feed stream; and,
passing the compressed feed stream to the first separation zone configured to separate a light ends stream from the compressed feed stream and provide the C2-4 olefins stream.

13. The process of claim 11 further comprising:
heating the C2-4 olefins stream with the partially hydrogenated effluent stream.

14. The process of claim 11 further comprising:
passing the partially hydrogenated effluent stream to a third separation zone having a fractionation column being configured to provide a C2− stream and a bottoms stream; and,
passing the bottoms stream from the third separation zone to the second separation zone.

15. The process of claim 14 further comprising:
passing the C2− stream to an acetylene conversion zone configured to convert acetylene in the C2− stream and provide an acetylene lean stream.

16. The process of claim 15 further comprising:
removing oxygenates from the acetylene lean stream to provide a purified stream.

17. The process of claim 16 further comprising:
passing the purified stream to a fourth separation zone configured to provide at least an ethylene stream and an ethane stream.

18. The process of claim 17 wherein the ethylene stream comprises less than or equal to 1 ppm of acetylene.

19. The process of claim 18 wherein the feed stream comprises an effluent from a reaction zone.

20. The process of claim 19 wherein the fourth separation zone also provides a recycle gas stream comprising hydrogen and ethylene, and the process further comprising:
recycling the recycle gas stream to the reaction zone.
